(19) United States
(12) Patent Application Publication
(10) Pub. No.: US 2025/0219656 A1
(43) Pub. Date: Jul. 3, 2025

(54) DATA COMPRESSION AND DATA DECOMPRESSION METHOD AND APPARATUS AND STORAGE MEDIUM

(71) Applicant: WUHAN UNITED IMAGING HEALTHCARE CO., LTD., Wuhan (CN)

(72) Inventors: KAI WEN LIU, Wuhan (CN); Yanke Cai, Wuhan (CN)

(21) Appl. No.: 19/002,621

(22) Filed: Dec. 26, 2024

(30) Foreign Application Priority Data
Dec. 27, 2023 (CN) 202311835757.8

(51) Int. Cl. H03M 7/30

(52) U.S. Cl. CPC H03M 7/6011; H03M 7/6005; H03M 7/70

(57) ABSTRACT

The present disclosure relates to data compression and data decompression methods and apparatuses, and storage media. The method includes: acquiring delay data from a plurality of focuses to different array elements during ultrasonic imaging; determining focus phase description information corresponding to the array elements based on the delay data; and compressing the focus phase description information corresponding to the array elements to obtain compressed delay data.

### CROSS REFERENCE TO RELATED APPLICATIONS

This application claims all benefits accruing under 35 U.S.C. § 119 to Chinese Patent Applications No. 2023118357578 filed on Dec. 27, 2023 in the China National Intellectual Property Administration, the content of which is hereby incorporated by reference.

### TECHNICAL FIELD

The present disclosure relates to the field of medical technologies, and in particular, to data compression and data decompression methods and apparatuses, and storage media.

### BACKGROUND

Ultrasonic imaging is widely applied to clinical medical diagnosis and examination. During real-time ultrasonic imaging, delay data from all focuses to different array elements is required to be transmitted to a computing device that executes a beamforming algorithm.

In the related art, in order to facilitate the transmission of the delay data, a large amount of delay data is compressed with a data compression method, and the compressed delay data is transmitted to the computing device.

### SUMMARY

The present disclosure provides data compression and data decompression methods and apparatuses, and storage media.

In a first aspect, the present disclosure provides a data compression method, including:
acquiring delay data from a plurality of focuses to different array elements during ultrasonic imaging;
determining focus phase description information corresponding to the array elements based on the delay data; and
compressing the focus phase description information corresponding to the array elements to obtain compressed delay data.

In an embodiment, determining the focus phase description information corresponding to the array elements based on the delay data includes:
acquiring, for any one of the array elements, an interpolation coefficient of delay data of all focuses corresponding to the array element; and
determining the focus phase description information corresponding to the array element according to the interpolation coefficient.

In an embodiment, the acquiring, for any one of the array elements, the interpolation coefficient of the delay data of all the focuses corresponding to the array element includes:
pre-determining, according to required image resolution and an error size, the interpolation coefficient of the delay data of all the focuses corresponding to the array element.

In an embodiment, the acquiring, for any one of the array elements, the interpolation coefficient of the delay data of all the focuses corresponding to the array element includes:
acquiring historical delay data most similar to the delay data of all the focuses corresponding to the array element, and determining an interpolation coefficient corresponding to the historical delay data to be the interpolation coefficient of the delay data of all the focuses corresponding to the array element.

In an embodiment, the focus phase description information includes phases of the focuses; and
determining the focus phase description information corresponding to the array element according to the interpolation coefficient includes:
quantizing the delay data of the focuses according to the interpolation coefficient, and determining a phase of each of the focuses; and
gathering the phase of each of the focuses to obtain the focus phase description information of the array element.

In an embodiment, gathering the phase of each of the focuses to obtain the focus phase description information of the array element includes:
taking a phase of a starting focus of the array element as an initial phase, and acquiring phases of other focuses after quantization;
generating a table indicating the phases of the plurality of focuses in order of the focuses; and
taking content of the table as the focus phase description information of the array element.

In an embodiment, the focus phase description information includes information on a number of focuses whose phases remain unchanged; and
determining the focus phase description information corresponding to the array element according to the interpolation coefficient includes:
acquiring delay increment data of adjacent focuses in the array element, and acquiring phases of the focuses corresponding to the array element;
if the delay increment data is within a preset range, determining phase change information of the adjacent focuses according to a mapping relationship between delay increment data and phase change information; and
determining the focus phase description information corresponding to the array element based on the phases of the focuses and the phase change information.

In an embodiment, acquiring the delay increment data of the adjacent focuses in the array element includes:
calculating a difference between the delay data of two adjacent focuses in the array element, and taking the difference as the delay increment data of the adjacent focuses in the array element.

In an embodiment, the preset range is greater than (N−1)/N*T and less than T, where T denotes an original sampling cycle of the delay data, and N denotes the interpolation coefficient.

In an embodiment, determining the focus phase description information corresponding to the array element based on the phases of the focuses and the phase change information includes:
sequentially determining, according to a focus sampling order, a number of focuses whose phases do not change, and gathering, according to the focus sampling order, the number of focuses whose phases do not change, to obtain the focus phase description information corresponding to the array element.

In an embodiment, determining the focus phase description information corresponding to the array element based on the phases of the focuses and the phase change information includes:
acquiring, for any one of the array elements, a phase of an initial focus corresponding to the array element;
calculating, based on the phase change information and the phases of the focuses, the information on the number of focuses whose phases remain unchanged; and
gathering the phase of the initial focus and the information on the number of focuses to obtain the focus phase description information corresponding to the array element.

In an embodiment, gathering the phase of the initial focus and the information on the number of focuses to obtain the focus phase description information corresponding to the array element includes:
putting the phase of the initial focus first in the focus phase description information; and
arranging, after the phase of the initial focus, the information on the number of focuses in order of sampling time, and gathering the arranged information, to obtain the focus phase description information of the array element.

In an embodiment, the focus phase description information includes a plurality of pieces of focus number information;
compressing the focus phase description information to obtain the compressed delay data includes:
sorting the plurality of pieces of focus number information in each piece of focus phase description information; and
compressing the sorted focus phase description information to obtain the compressed delay data.

In an embodiment, the method further includes:
acquiring initial focus delay data of each of the array elements; and
transmitting the initial focus delay data and the compressed delay data to a computing device.

In a second aspect, the present disclosure further provides a data decompression method, including:
receiving a plurality of pieces of initial focus delay data and compressed delay data; the compressed delay data being obtained by compressing focus phase description information corresponding to a plurality of array elements; the focus phase description information being determined based on a plurality of pieces of delay data; and the delay data being delay data from a plurality of focuses to different array elements during ultrasonic imaging; and
decompressing the compressed delay data to obtain the delay data from the plurality of focuses to the different array elements during the ultrasonic imaging.

In an embodiment, the method further includes: performing beamforming based on the obtained delay data from the plurality of focuses to the different array elements during the ultrasonic imaging to obtain an ultrasonic image.

In a third aspect, the present disclosure further provides a data compression apparatus, the apparatus including a memory and a processor, the memory storing a computer program, and the processor, when executing the computer program, implementing the following processing:
acquiring delay data from a plurality of focuses to different array elements during ultrasonic imaging;
determining focus phase description information corresponding to the array elements based on the delay data; and
compressing the focus phase description information corresponding to the array elements to obtain compressed delay data.

In a fourth aspect, the present disclosure further provides a data decompression apparatus, the apparatus including a memory and a processor, the memory storing a computer program, and the processor, when executing the computer program, implementing the following processing:
receiving a plurality of pieces of initial focus delay data and compressed delay data; the compressed delay data being obtained by compressing focus phase description information corresponding to a plurality of array elements; the focus phase description information being determined based on a plurality of pieces of delay data; and the delay data being delay data from a plurality of focuses to different array elements during ultrasonic imaging; and
decompressing the compressed delay data to obtain the delay data from the plurality of focuses to the different array elements during the ultrasonic imaging.

In a fifth aspect, the present disclosure further provides a non-transitory computer-readable storage medium. The non-transitory computer-readable storage medium has a computer program stored therein. Content in any one of the embodiments of the data compression method in the first aspect is implemented when the computer program is executed by a processor.

In a sixth aspect, the present disclosure further provides a non-transitory computer-readable storage medium. The non-transitory computer-readable storage medium has a computer program stored therein. Content in any one of the embodiments of the data decompression method in the first aspect is implemented when the computer program is executed by a processor.

In a seventh aspect, the present disclosure further provides a computer program product. The computer program product includes a computer program. Content in any one of the embodiments of the data compression method in the first aspect is implemented when the computer program is executed by a processor.

According to the data compression and data decompression methods and apparatuses, and storage media above, delay data from a plurality of focuses to different array elements during ultrasonic imaging is acquired, focus phase description information corresponding to the array elements is determined based on the delay data, and the focus phase description information is compressed to obtain compressed delay data. According to the method, a plurality of pieces of delay data are analyzed to determine phases of the focuses corresponding to each array element and determine phase description information, and the phase description information is directly compressed, which reduces complexity of a calculation process, thereby reducing complexity of data compression. On the basis of reducing the complexity of data compression, complexity of the data decompression process is also inevitably reduced. At the same time, an amount of compressed data during compression is also greatly reduced.

### BRIEF DESCRIPTION OF THE DRAWINGS

FIG. 1 is a diagram of an application environment of a data compression method according to an embodiment;

FIG. 2 is a first schematic flowchart of the data compression method according to an embodiment;

FIG. 3 is a second schematic flowchart of the data compression method according to an embodiment;

FIG. 4 is a third schematic flowchart of the data compression method according to an embodiment;

FIG. 5 is a fourth schematic flowchart of the data compression method according to an embodiment;

FIG. 6 is a schematic diagram of phase change information according to an embodiment;

FIG. 7 is a fifth schematic flowchart of the data compression method according to an embodiment;

FIG. 8 is schematic diagram of a curve of focus phase description information according to an embodiment;

FIG. 9 is a sixth schematic flowchart of the data compression method according to an embodiment;

FIG. 10 is a seventh schematic flowchart of the data compression method according to an embodiment;

FIG. 11 is an eighth schematic flowchart of the data compression method according to an embodiment;

FIG. 12 is a schematic diagram of delay errors according to an embodiment;

FIG. 13 is a schematic diagram of sorting errors according to an embodiment;

FIG. 14 is a first schematic flowchart of a data decompression method according to an embodiment;

FIG. 15 is a second schematic flowchart of the data decompression method according to an embodiment; and

FIG. 16 is a structural block diagram of a data compression apparatus according to an embodiment.

### DETAILED DESCRIPTION

In order to make the objective, technical solutions, and advantages of the present disclosure clearer, the present disclosure will be further described in detail below with reference to the accompanying drawings and embodiments. It should be understood that specific embodiments described herein are only intended to explain the present disclosure and are not intended to limit the present disclosure.

Prior to detailed introduction to the technical solutions of the present disclosure, the background of the present disclosure will be briefly introduced first.

Ultrasonic imaging is a technology widely applied to clinical medical diagnosis and examination. A beamforming technology is a common technical means in the ultrasonic imaging. In the beamforming technology, delay data from all focuses to different array elements is required to be acquired. The delay data refers to time delay information required for ultrasonic waves emitted from different focuses to reach each array element. The delay data is used to control emission time of each array element in an ultrasonic probe array to form a focused ultrasound beam at a specific depth. By accurately calculating a delay of each array element, the emitted ultrasonic waves can be phase-synchronized within a predetermined depth, thereby enhancing resolution and contrast of imaging. During real-time ultrasonic imaging, the delay data is transmitted to a computing device that executes a beamforming algorithm. However, this method requires a higher data transmission bandwidth, and the computing device needs an enough storage space to store such delay data and then reads the delay data from the storage space to perform beamforming.

In the related art, in order to facilitate the transmission of the delay data, a large amount of delay data is mainly compressed with a data compression method, and the compressed delay data is transmitted to the computing device.

However, with the development of the technology, a number of array elements of an ultrasonic probe continues to increase, a sampling rate of an ultrasonic system continues to increase, and a delay error is required to be as small as possible. The above factors make the amount of delay data used for beamforming increasingly large, making it more complex to compress the delay data.

Therefore, with respect to the above problems, the present disclosure provides a data compression method, which can reduce an amount of data during compression of delay data, thereby solving the problem of higher complexity of data compression.

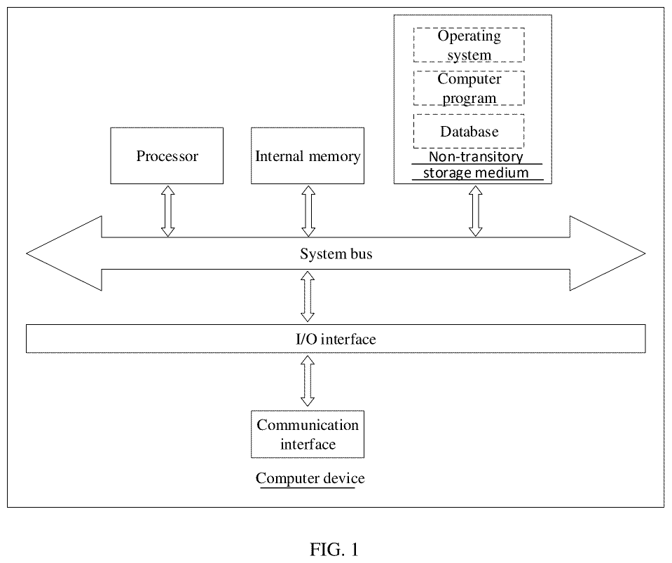

The data compression method provided in embodiments of the present disclosure is applicable to an application environment as shown in FIG. 1. For example, the computer device may be a server, a personal computer, a notebook computer, a smartphone, a tablet computer, a smart mobile phone, or the like. The computer device may include a processor, a memory, and a network interfaces connected through a system bus or wirelessly. The processor of the computer device is configured to provide computing and control capabilities. The memory of the computer device includes a non-transitory storage medium and an internal memory. The non-transitory storage medium stores an operating system, a computer program, and a database. The internal memory provides an environment for running of the operating system and the computer program in the non-transitory storage medium. The database of the computer device is configured to store data during data compression. The network interface of the computer device is configured to communicate with an external terminal through a network connection. The computer program is executed by the processor to implement a data compression method. The computer device may be implemented by a standalone computer device or a computer device cluster formed by a plurality of computer devices. It is to be noted that the memory of the computer device is not limited to the above memory, and may also include a high-speed random access memory, a transitory solid-state memory, and the like. In addition, the composition structure of the computer equipment is not limited to the above, and some components may alternatively be added or omitted.

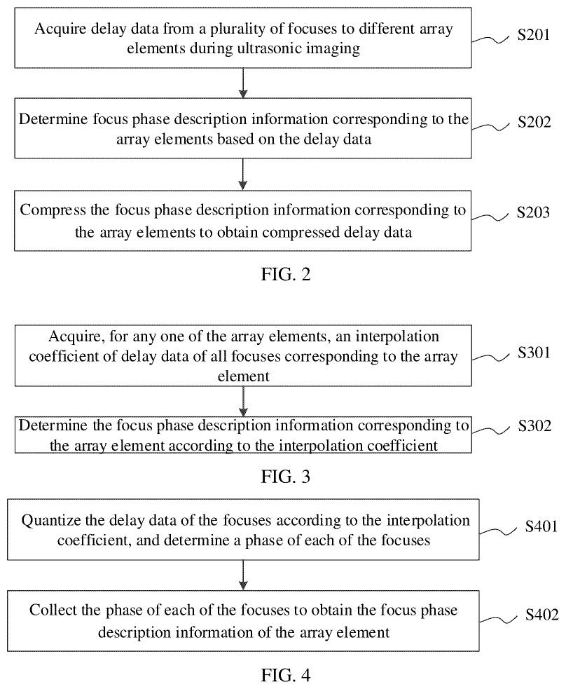

In an embodiment, as shown in FIG. 2, a data compression method is provided. For example, the method is applied to the computer device in FIG. 1, including the following steps.

In S201, delay data from a plurality of focuses to different array elements during ultrasonic imaging is acquired.

An ultrasonic receiving beamforming technology is a technology that improves quality and resolution of ultrasonic imaging by combining received signals from a plurality of sensors or array elements. In the ultrasonic receiving beamforming technology, since each array element may receive ultrasonic echoes returned from a plurality of focuses on a target object, the above delay data refers to data from the plurality of focuses on the target object to different array elements.

In the embodiments of the present disclosure, the computer device may determine delay data of a plurality of array elements according to parameter information and sampling parameter information of the array elements. For example, the parameter information of the array elements may be sizes of the array elements, spacings between the array elements, and the like. The sampling parameter may be a sampling rate of an analog-to-digital converter, or the like.

Alternatively, the computer device may acquire the delay data from the plurality of focuses to the different array elements from a memory of delay data. In this embodiment, the manner of acquiring the delay data from the plurality of focuses to the different array elements during the ultrasonic imaging is not limited.

In S202, focus phase description information corresponding to the array elements is determined based on the delay data.

The focus phase description information refers to characteristic information of phases of the plurality of focuses. For example, the focus phase description information may be phases of the plurality of focuses or regular information of phase changes between the plurality of focuses. It is to be noted that an ultrasonic image is formed by a plurality of receiving lines, and each piece of focus phase description information corresponds to all phase description information on one receiving line. The receiving line is an ultrasonic path of the ultrasonic image in an axial direction. For example, a number of the receiving line is N, and each array element corresponds to N pieces of focus phase description information. The receiving line refers to a virtual line along a depth direction of an ultrasonic imaging region. Beamforming focuses are distributed on each line.

In the embodiments of the present disclosure, for each array element, the computer device may analyze delay data of the plurality of focuses to the array element to determine a phase of each focus. Moreover, a phase characteristic of each focus is described and/or a relationship of phase characteristics between the plurality of focuses is described, to obtain the focus phase description information corresponding to the array element. Alternatively, the computer device may further input the delay data from the plurality of focuses to the array element into a description information determination model, and the delay data is analyzed by the description information determination model to determine the focus phase description information corresponding to the array element. In this embodiment, the manner of determining the focus phase description information corresponding to the array elements based on the delay data is not limited.

In S203, the focus phase description information corresponding to the array elements is compressed to obtain compressed delay data.

In the embodiments of the present disclosure, the computer device may compress a plurality of pieces of focus phase description information by using a data compression algorithm, compress the plurality of pieces of focus phase description information into one file, and take the file as the compressed delay data. For example, the data compression algorithm may be a Huffman coding algorithm, a dictionary coding algorithm, a model-based compression algorithm, or the like.

In the above data compression method, delay data from a plurality of focuses to different array elements during ultrasonic imaging is acquired, focus phase description information corresponding to the array elements is determined based on the delay data, and the focus phase description information corresponding to the array elements is compressed to obtain compressed delay data. According to the method, a plurality of pieces of delay data are analyzed to determine phases of a plurality of focuses corresponding to each array element and determine phase description information of the focuses, and the phase description information is directly compressed, which reduces complexity of a calculation process, thereby reducing complexity of data compression. On the basis of reducing the complexity of data compression, complexity of the data decompression process is also inevitably reduced. At the same time, an amount of compressed data during compression is also greatly reduced.

On the basis of the above embodiments, in this embodiment, related content of “determining focus phase description information corresponding to the array elements based on the delay data” in step S202 in FIG. 2 is described. As shown in FIG. 3, as a non-limiting example, step S202 above may include the following content.

In S301, for any one of the array elements, an interpolation coefficient of delay data of all focuses corresponding to the array element is acquired.

For any one of the array elements, due to a long original sampling cycle, delay data of at least some focuses corresponding to the array element may not be at a sampling point. In this way, it is inaccurate to directly determine phases of such focuses. In order to ensure accuracy of the phases of the focuses corresponding to the array element, there is a need to select an appropriate interpolation coefficient to interpolate the delay data of all the focuses.

In the embodiments of the present disclosure, the computer device may pre-determine, according to required image resolution and an error size, the interpolation coefficient of the delay data of all the focuses corresponding to the array element. For example, the interpolation coefficient may be 4 times interpolation or 8 times interpolation. The interpolation coefficient is an integer. Alternatively, the computer device may acquire historical delay data most similar to the delay data of all the focuses corresponding to the array element, and determine an interpolation coefficient corresponding to the historical delay data to be the interpolation coefficient of the delay data of all the focuses corresponding to the array element.

In S302, the focus phase description information corresponding to the array element is determined according to the interpolation coefficient.

In the embodiments of the present disclosure, the computer device may interpolate the delay data of all the focuses based on the interpolation coefficient, and acquire a phase corresponding to each focus according to the delay data of the focus. After the phases of all the focuses are acquired, characteristics of the phases of all the focuses are described, to obtain the focus phase description information corresponding to the array element.

In the above data compression method, for any one of the array elements, an interpolation coefficient of delay data of all focuses corresponding to the array element is acquired, and the focus phase description information corresponding to the array element is determined according to the interpolation coefficient. According to the method, for any array element, an interpolation coefficient suitable for the delay data of all the focuses can be selected, so that the phase of each focus can be accurately acquired based on the interpolation coefficient, thereby accurately acquiring the focus phase description information corresponding to the array element.

On the basis of the above embodiments, the focus phase description information includes phases of the focuses, and related content of “determining the focus phase description information corresponding to the array element according to the interpolation coefficient” in step S302 in FIG. 3 is described. As shown in FIG. 4, as a non-limiting example, step S302 above may include the following content.

In S401, the delay data of the focuses is quantized according to the interpolation coefficient, and a phase of each of the focuses is determined.

In the embodiments of the present disclosure, the computer device may quantize a plurality of pieces of delay data according to the interpolation coefficient, to divide the delay data of each focus in more detail. In this way, a position of each piece of delay data in a sampling cycle after interpolation can be accurately determined, so that the phase of the focus corresponding to the delay data can be determined based on the position. Exemplarily, if the original sampling cycle of the delay data is T, after interpolation, A denotes a coarse delay of the delay data under the original sampling cycle. Coarse delay refers to delay data in units of an original sampling cycle. In specific calculation, coarse delay=floor (ideal delay data/sampling cycle), and floor represents an operation for downward integer. A is in units of T. That is, A is data in units of the original sampling cycle. For example, when the delay data is 103 and the original sampling cycle is 10, coarse delay data of the delay data in the original sampling cycle may be expressed as 10. The sampling cycle after interpolation is T/N. B denotes phase, and is data in units of the sampling cycle after interpolation. Ideal delay data may be expressed as A*T+B*T/N. If an interpolation manner is a polyphase filter interpolation algorithm, the original sampling cycle may be divided into N phases. That is, a phase change range of B is 0, 1, 2, . . . and (N−1). In this case, the interpolation coefficient is N, that is, after interpolation, N−1 new sampling points are interpolated into each interval (the original sampling cycle) whose length is T, so that the original sampling cycle is divided into N phases of 0 to (N−1). According to the above formula, the phase B can be determined as =round ((delay data−coarse delay*sampling cycle)/(sampling cycle/interpolation coefficient)), round representing rounding operation.

In S402, the phase of each of the focuses is gathered (summarized) to obtain the focus phase description information of the array element.

In the embodiments of the present disclosure, after the phase of each focus is acquired, the computer device may combine the phases of the plurality of focuses into a table, and take content of the table as the focus phase description information of the array element. In the process, a phase of a starting focus of the array element may be taken as an initial phase, phases of other focuses after quantization are acquired, and the phases of the focuses are sorted in the table in the order of the focuses, i.e., in the order of sampling time or the order of sampling depth.

In the above data compression method, the delay data of the focuses is quantized according to the interpolation coefficient, a phase of each of the focuses is determined, and the phase of each of the focuses is determined to be the focus phase description information of the array element. According to the method, the plurality of pieces of delay data can be further quantized through the interpolation coefficient. In this way, the phase of each focus can be more accurately acquired, thereby obtaining the focus phase description information of the array element.

On the basis of the above embodiments, the focus phase description information includes information on a number of focuses whose phases remain unchanged, and related content of “determining the focus phase description information corresponding to the array element according to the interpolation coefficient” in step S302 in FIG. 3 is described in this embodiment. As shown in FIG. 5, as a non-limiting example, step S302 above may include the following content.

In S501, delay increment data of adjacent focuses in the array element is acquired, and phases of the focuses corresponding to the array element is acquired.

In the embodiments of the present disclosure, for any array element, the array element corresponds to a plurality of focuses, and each focus corresponds to one piece of delay data. For two adjacent focuses of any group, the computer device may calculate a difference between the delay data of the two adjacent focuses, and take the difference as the delay increment data of the adjacent focuses.

In addition, after the delay data of all the focuses is quantized, a position of each piece of delay data in the sampling cycle after interpolation can be determined. Moreover, based on a corresponding relationship between positions and phases, the phase of each focus can be accurately acquired, and the phase of each focus is determined to be the phases of the focuses corresponding to the array element.

In S502, if the delay increment data is within a preset range, phase change information of the adjacent focuses is determined according to a mapping relationship between delay increment data and phase change information.

Since the focus phase description information includes the information on the number of focuses whose phases remain unchanged, the delay increment data of two adjacent focuses is required to meet a preset condition. The adjacent focuses can fall within the original sampling cycle one by one only when the preset condition is met. In this case, the phase of the delay data can conform to a preset change rule.

In the embodiments of the present disclosure, the computer device may judge each piece of delay increment data to determine whether all the delay increment data is within the preset range. For example, the preset range may be [(N−1)/N*T, T]. T denotes the original sampling cycle of the delay data. For any array element, a delay increment of adjacent focuses is greater than (N−1)/N*T and gradually approaches T.

Exemplarily, in order to ensure that the delay increment data between at least some focus points is within a preset range, it is preset that the distance between adjacent focuses=C*(T/2), where C is the speed of sound and T is the sampling cycle. In a case where the distance between adjacent focuses satisfies the above range, the delay increment data between subsequent focuses starting from an initial focus (which will be described later in detail) is in the preset range.

Further, if all the delay increment data in one array element is within the preset range, adjacent focuses can fall within the original sampling cycle one by one. Then, there is a corresponding rule for phase changes of two adjacent focuses. The computer device may analyze the plurality of pieces of delay increment data according to the mapping relationship between delay increment data and phase change information, to determine phase change information of the adjacent focuses. For example, if the delay increment of adjacent focuses is greater than (N−1)/N*T and gradually approaches T, the mapping relationship may be determined to be that phases of the adjacent focuses remain unchanged or decrease by 1 phase. A phase change from 0 to (N−1) is also considered to be “decrease by 1 phase”. The phase change information refers to information about how the phase of the adjacent focuses satisfying the preset change rule changes. That is, as long as a delay increment data between adjacent focuses is greater than (N−1)/N*T and gradually approaches T, the phase between the adjacent focuses satisfies the foregoing mapping relationship. According to the mapping relationship, only the number X of adjacent focuses whose phases are kept unchanged may be recorded. Since X indicates the number of adjacent focuses whose phases are kept unchanged, The (X+1)-th focus starting from the current focus is the focus with one phase reduced, so that the phase of the (X+1)-th focus can be obtained, In other words, the phase of the (X+1)-th focus is one phase reduced from the phase of the current focus.

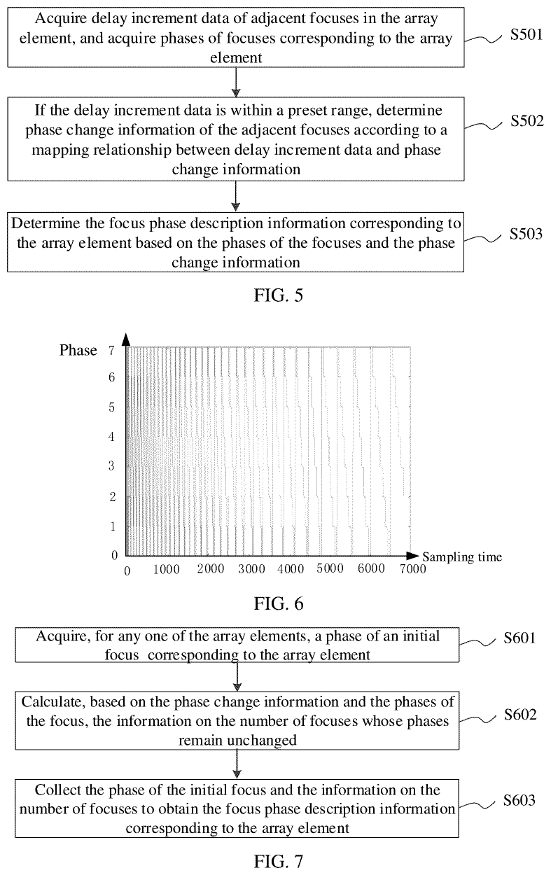

FIG. 6 is a schematic diagram of phase change information. In the figure, the X axis represents sampling points, and the ordinate represents phases. When the interpolation coefficient is 8 times interpolation, the original sampling cycle is divided into 8 phases. As depths of the focuses increase, the phases of the focuses cycle according to the following trend: the initial phase remains unchanged or decreases by 1. For example, when the phase of the first delay data is 0, the phase of the second delay data may remain unchanged or decrease by 1. The phase of the second delay data is still 0, or the phase of the second delay data changes to 7. In addition, as the depths increase, the number of focuses whose phases remain unchanged increases.

In S503, the focus phase description information corresponding to the array element is determined based on the phases of the focuses and the phase change information.

In the embodiments of the present disclosure, after all the phases of the focuses and the phase change information are acquired, the computer device may sequentially determine, according to a focus sampling order, a number of focuses whose phases do not change, and gather (summarize), according to the focus sampling order, the number of focuses whose phases do not change, to obtain the focus phase description information corresponding to the array element.

In the above data compression method, delay increment data of adjacent focuses in the array element is acquired, phases of the focuses corresponding to the array element is acquired, if the delay increment data is within a preset range, phase change information of the adjacent focuses is determined according to a mapping relationship between delay increment data and phase change information, and the focus phase description information corresponding to the array element is determined based on the phases of the focuses and the phase change information. According to the method, by judging the delay increment data of adjacent focuses, whether the adjacent focuses conform to a phase change rule is determined, so that the phase change information of the focuses can be accurately determined based on the phase change rule. Then, the information on the number of focuses whose phases remain unchanged is accurately acquired according to the phases of the focuses and the phase change information, so that more accurate focus phase description information can be obtained.

On the basis of the above embodiments, in this embodiment, related content of “determining the focus phase description information corresponding to the array element based on the phases of the focuses and the phase change information” in step S503 in FIG. 5 is described. As shown in FIG. 7, as a non-limiting example, step S503 above may include the following content.

In S601, for any one of the array elements, a phase of an initial focus corresponding to the array element is acquired.

In the embodiments of the present disclosure, for any array element, the computer device may calculate, based on the time delay data of the focus relative to the array element, time delay increment data of adjacent focuses along the depth direction on a receiving line. Exemplarily, for a certain receiving line, the delay data of the first focusing point relative to the array element is subtracted from the delay data of the second focusing point relative to the array element where the receiving line is located, so as to obtain the first delay increment data; subtracting delay data of a second focus relative to the array element from delay data of a third focus relative to the array element to obtain a second delay difference, and by analogy, when there are R focuses on the receiving line, R−1 pieces of delay increment data relative to the array element can be obtained. Starting from the first delay difference, when the S-th delay increment data is greater than or equal to (N−1)/N*T, where N is an interpolation coefficient and T is a sampling period, it is determined that the S-th focus is an initial focus of the receiving line relative to the array element, and the phase of the focus is determined as the phase of the initial focus.

In S602, the information on the number of focuses whose phases remain unchanged is calculated based on the phase change information and the phases of the focuses.

In the embodiments of the present disclosure, the computer device may determine, from a plurality of phases of the focuses according to the phase change rule, information on a number of focuses whose phases are the same. It should be emphasized that in the process, calculation is performed according to sampling time of the delay data of the focuses, instead of calculating a number of focuses with unchanged phases in all the phases of the focuses. For example, from the first focus, the number of focuses whose phases are 3 is 5, phases of the sixth and seventh focuses change to 2, and the number of focuses whose phases are 2 is 2. By analogy, the information on the number of focuses whose phases remain unchanged is obtained according to the order.

In S603, the phase of the initial focus and the information on the number of focuses whose phases remain unchanged are gathered (summarized) to obtain the focus phase description information of the array element.

In the embodiments of the present disclosure, the computer device may put the phase of the initial focus first in the focus phase description information, and then arrange, after the phase of the initial focus, the information on the number of focuses in order of sampling time. Moreover, the arranged information is gathered (summarized) to obtain the focus phase description information of the array element. The focus phase description information may be obtained with reference to Table 1. In Table 1, an array element 1 and an array element 2 are described respectively by taking the interpolation coefficient as 8 times interpolation. Taking the array element 1 as an example, the first row is a phase of the initial phase, and the phase of the initial focus is 3. Rows 2 to 7 are numbers of focuses that maintain a same phase. As can be seen, the number of focuses whose phases are 3 is 7, the number of focuses whose phases are 2 is 9, the number of focuses whose phases are 1 is 8, the number of focuses whose phases are 0 is 9, the number of focuses whose phases are 7 is 10, and the number of focuses whose phases are 6 is 11.

TABLE 1

| Number of rows | Array element 1 | Array element 2 |
|---|---|---|
| 1 | 3 | 2 |
| 2 | 7 | 5 |
| 3 | 9 | 6 |
| 4 | 8 | 7 |
| 5 | 9 | 8 |
| 6 | 10 | 10 |
| 7 | 11 | 11 |

FIG. 8 is a schematic diagram of a curve of focus phase description information. The figure is generated based on the focus phase description information of the array element 1. As can be seen from the figure, the values related to unchanged phases show an increasing pattern as a whole, and after the curve increases to a certain degree, the values suddenly decrease, it is because the number of the focuses is limited.

In the above data compression method, for any one of the array elements, a phase of an initial focus corresponding to the array element is acquired, the information on the number of focuses whose phases remain unchanged is calculated based on the phase change information and the phases of the focuses, and the phase of the initial focus and the information on the number of focuses are gathered (summarized) to obtain the focus phase description information of the array element. According to the method, the information on the number of focuses whose phases remain unchanged can be determined from phases of the focuses according to the phase change information and the phases of the focuses, and the focus phase description information of the array element can be accurately acquired based on the phase of the initial focus.

On the basis of the above embodiments, the focus phase description information includes a plurality of pieces of focus number information; and related content of “compressing the focus phase description information to obtain compressed delay data” in step S203 in FIG. 2 is described in this embodiment. As shown in FIG. 9, as a non-limiting example, step S203 above may include the following content.

In S701, the plurality of pieces of focus number information in each piece of focus phase description information are sorted.

In the embodiments of the present disclosure, after the focus phase description information is acquired, the plurality of pieces of focus number information are a monotonically increasing change on the whole, but there may be fluctuations. For example, for the array element 1 in Table 1, the third row is 9, and the fourth row is 8. This is the situation where there is a fluctuation. The computer device may exchange the two orders so that the plurality of pieces of focus number information in the focus phase description information satisfy a monotonically increasing rule. The sorted focus phase description information of the array element 1 is represented through Table 2.

TABLE 2

| Number of rows | Array element 1 |
|---|---|
| 1 | 3 |
| 2 | 7 |
| 3 | 8 |
| 4 | 9 |
| 5 | 9 |
| 6 | 10 |
| 7 | 11 |

In S702, the plurality of pieces of sorted focus phase description information are compressed to obtain the compressed delay data.

In the embodiments of the present disclosure, after the plurality of pieces of sorted focus phase description information are acquired, from the second row, the computer device may calculate a difference between adjacent rows and obtain difference change information. In a specific example, data of the first two rows may be retained, and a difference value between the data of the row and data of the previous row is calculated from the third row as difference value change information; for example, a difference 1 is obtained by subtracting the second row of numerical values 7 from the third row of numerical values 8, and a difference 1 is obtained by subtracting the third row of numerical values 8 from the fourth row of numerical values 9, as difference change information. The computer device may encode the difference change information to obtain the compressed delay data. It may be found based on the difference change information that the difference is generally 0 or 1 in a shallower region of each array element, and the difference is generally greater than or equal to 2 in a deeper region. Therefore, for the shallower region, the computer device may perform encoding by 1 bit. For deeper region, the computer device may select different numbers of bits for encoding according to a range of differences. At the same time, fields are provided to describe a number of differences encoded with 1 bit, as well as an encoding scheme and a number of differences encoded with 2 or more bits. After the encoding is completed, the compressed delay data is obtained.

In the above data compression method, the plurality of pieces of focus number information in each piece of focus phase description information are sorted, and the sorted focus phase description information is compressed to obtain the compressed delay data. According to the method, the plurality of pieces of focus number information is processed by sorting, so that the focus phase description information conforms to a certain rule. In this way, it is more convenient to compress the focus phase description information, thereby greatly reducing complexity of the compression.

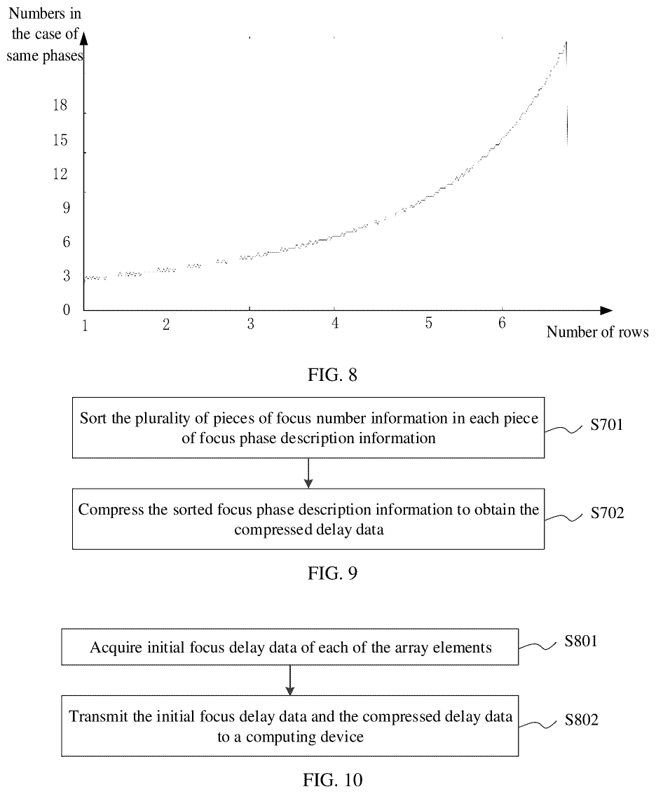

On the basis of the above embodiments, related content of data transmission of the compressed delay data is described in this embodiment. As shown in FIG. 10, as a non-limiting example, the above method may further include the following content.

In S801, initial focus delay data of each of the array elements is acquired.

In the embodiments of the present disclosure, the computer device may acquire an initial focus of each array element in the manner in step S601, and then acquire the initial focus delay data of each array element from the plurality of pieces of delay data.

In S802, the initial focus delay data and the compressed delay data are transmitted to a computing device.

In the embodiments of the present disclosure, during data transmission, there is a need to ensure that the computing device may accurately calculate the delay data from the plurality of focuses to the different array elements based on received information. Therefore, during the data transmission, the computer device may transmit the initial focus delay data corresponding to each array element and the compressed delay data to the computing device. It should be emphasized that the initial focus delay data may alternatively be replaced with a position of a sampling point corresponding to the initial focus. The initial focus refers to an i^{th} sampling point prior to interpolation.

In the above data compression method, initial focus delay data of each array element is acquired, and the initial focus delay data and the compressed delay data are transmitted to a computing device. According to the method, during data transmission, the initial focus delay data and the compressed delay data are transmitted to the computing device, so that the computing device can accurately acquire the delay data from the plurality of focuses to the different array elements according to the received data.

On the basis of the above embodiments, the data compression method further includes: sending the compressed delay data to a computing device, and instructing the computing device to perform beamforming based on the compressed delay data so as to obtain an ultrasonic image.

Specifically, the computing device firstly performs decompression processing on compressed delay data to obtain delay data from the plurality of focuses to different array elements during ultrasonic imaging, i.e., the decompressed delay data, then performs beamforming on the decompressed delay data to obtain a signal of a target region, and performs signal processing on the signal of the target region obtained after beamforming to obtain an ultrasonic image.

Still further, the data compression method may further include adjusting at least one of position and parameter of an ultrasound probe based on the obtained ultrasound image, or sending an instruction for adjusting at least one of the position and the parameter of the ultrasound probe to the ultrasound probe.

Specifically, when the ultrasonic image indicates that the focusing of the ultrasonic beam is inaccurate, that is, the result after beamforming indicates that the ultrasonic beam is not focused on an expected target depth or area, the position and/or parameter of the ultrasonic probe may be adjusted, or an instruction for adjusting the position and/or parameter of the ultrasonic probe may be sent to the ultrasonic probe, so as to ensure that the beam can be continuously focused on the target. The parameter of the ultrasonic probe is a parameter applied to the ultrasonic probe when the ultrasonic probe is in operation, for example, including an operating frequency, a transmission power or the like of the ultrasonic probe. Adjusting the position and/or parameters of the ultrasound probe can optimize the effects of beamforming.

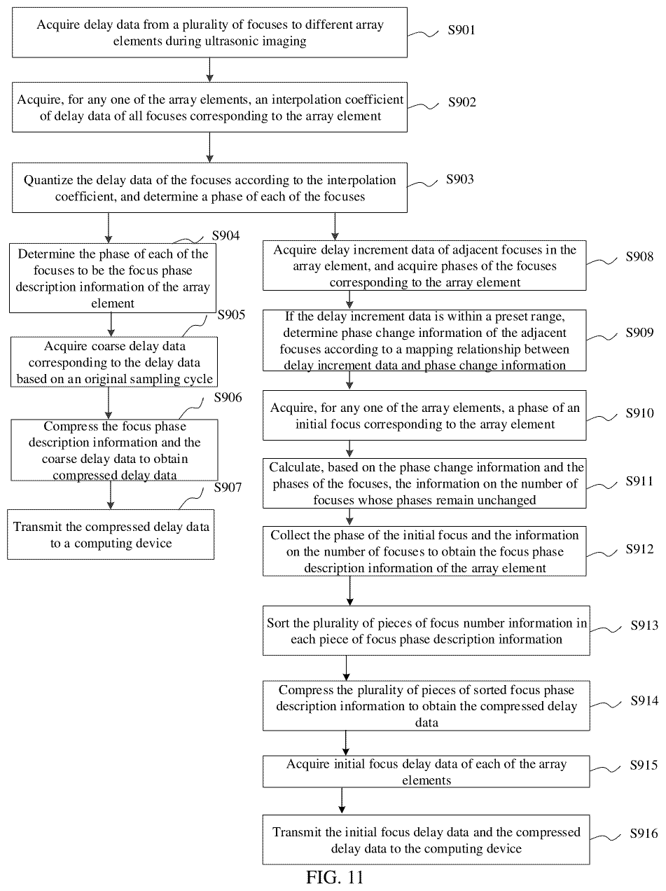

As a specific embodiment of the present disclosure, as shown in FIG. 11, the data compression method includes the following steps.

In S901, delay data from a plurality of focuses to different array elements during ultrasonic imaging is acquired.

In S902, for any one of the array elements, an interpolation coefficient of delay data of all focuses corresponding to the array element is acquired.

In S903, the delay data of the focuses is quantized according to the interpolation coefficient, and a phase of each of the focuses is determined.

In S904, the phase of each of the focuses is determined to be the focus phase description information of the array element.

In S905, coarse delay data corresponding to the delay data is acquired based on an original sampling cycle.

In S906, the focus phase description information and the coarse delay data are compressed to obtain compressed delay data.

In S907, the compressed delay data is transmitted to a computing device.

In S908, delay increment data of adjacent focuses in the array element is acquired, and phases of the focuses corresponding to the array element is acquired.

In S909, if the delay increment data is within a preset range, phase change information of the adjacent focuses is determined according to a mapping relationship between delay increment data and phase change information.

In S910, for any one of the array elements, a phase of an initial focus corresponding to the array element is acquired.

In S911, the information on the number of focuses whose phases remain unchanged is calculated based on the phase change information and the phases of the focuses.

In S912, the phase of the initial focus and the information on the number of focuses are gathered to obtain the focus phase description information of the array element.

In S913, the plurality of pieces of focus number information in each piece of focus phase description information are sorted.

In S914, the plurality of pieces of sorted focus phase description information are compressed to obtain the compressed delay data.

In S915, initial focus delay data of each of the array elements is acquired.

In S916, the initial focus delay data and the compressed delay data are transmitted to the computing device.

It is to be noted that step S901 to step S907 are the first manner. In the manner, the compressed data transmitted to the computing device includes a phase and coarse delay data of each focus, and the computing device may infer the delay data of the focus according to the phase and the coarse delay data of each focus. For example, if the coarse delay data is 10, the original sampling cycle is 8, the interpolation coefficient is 8, and the phase of any focus is 3, according to delay data=coarse delay data*original sampling cycle+phase*(original sampling cycle/interpolation coefficient), it may be inferred that the delay data is 83. Step S908 to step S916 are the second manner. In the manner, the compressed data transmitted to the computing device includes a focus phase change rule and delay data of the initial focus, and the computing device may infer the delay data of the focus according to the delay data of the initial focus and the focus phase change rule. This is because, in the second manner, the coarse delay data also satisfies a certain rule. A change rule of the coarse delay data is related to phase changes. As a depth increases, if a phase of a current focus is N−1 and a phase of a previous focus is 0, coarse delay data of the current focus is the same as coarse delay data of the previous focus. Otherwise, the coarse delay data of the current focus is 1 greater than the coarse delay data of the previous focus. Therefore, in the second manner, the coarse delay data may be obtained from phase data and initial focus delay, and in the second manner, there is no need to transmit the coarse delay data to the computing device. For example, when the delay data of the first focus is 83, the phase of the first focus is 3, and it is determined based on the focus phase change rule that the phase of the second focus is still 3, the coarse delay data of the second focus is greater by 1 than the coarse delay of the first focus, and therefore the coarse delay of the second focus point is equal to 10+1=11, and the second delay data is 11*8+3*(8/8)=91.

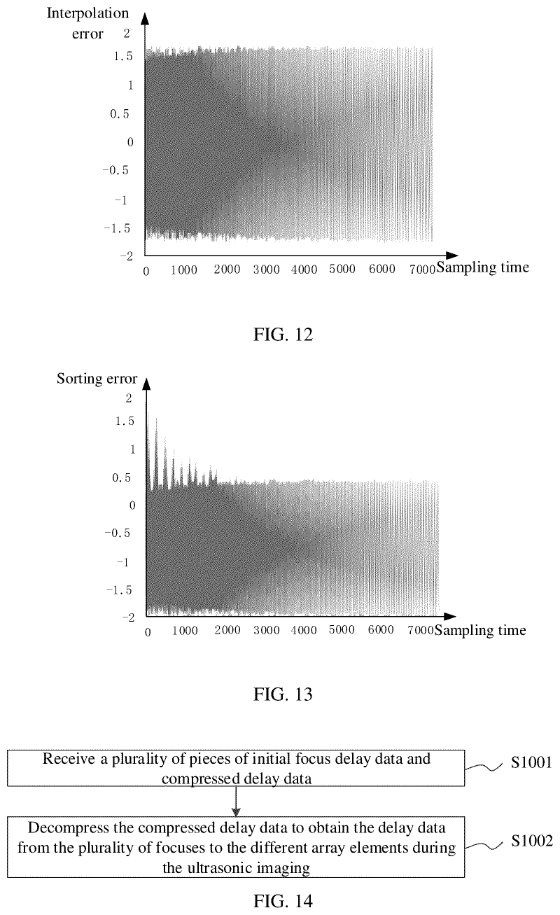

In addition, considering that the interpolation process may bring interpolation errors in the process of determining the phase corresponding to the delay data and compressing the phase description information, the sorting process may also bring sorting errors. The interpolation error means that a quantization unit used during the quantization is a sampling cycle after interpolation. Therefore, the error is negatively related to the interpolation coefficient. That is, a larger interpolation coefficient indicates a smaller error. FIG. 12 is a schematic diagram showing interpolation errors. The X axis represents sampling time, and the Y axis represents interpolation errors. When rounding is used during the quantization, an absolute value of the quantization error is approximately equal to T/N/2. Here, the quantization error is obtained by comparing the decompressed delay data with the original delay data. As shown in FIG. 12, when the sampling cycled is 25 ns and the interpolation coefficient is 8, a peak-to-peak value of the quantization error is less than 25/8=3.125 ns, and the absolute value is less than 3.125/2=1.5625 ns. In order to solve the interpolation error, in the embodiments of the present disclosure, the interpolation coefficient may be appropriately increased to compensate for the interpolation error.

FIG. 13 is a schematic diagram showing sorting errors. The X axis represents sampling time, and the Y axis represents sorting errors. As can be seen from FIG. 13, in a shallower region, a phase change is relatively drastic, while in a deeper region, there are more focuses whose phases remain unchanged. Therefore, an error introduced during the sorting is mainly reflected in the shallower region. As can be seen from the figure, the absolute value of the error is still small and does not affect parsing and calculation processes of the delay data by the computing unit.

In an embodiment, as shown in FIG. 14, a data decompression method is provided. For example, the method is applied to the computing device that executes a beamforming algorithm. The method includes the following steps.

In S1001, a plurality of pieces of initial focus delay data and compressed delay data are received, wherein the compressed delay data is obtained by compressing focus phase description information corresponding to a plurality of array elements, the focus phase description information is determined based on a plurality of pieces of delay data, and the delay data is delay data from a plurality of focuses to different array elements during ultrasonic imaging.

In the embodiments of the present disclosure, when the compressed delay data is compressed in the computer device and the compressed delay data and a plurality of pieces of initial focus delay data are transmitted to the computing device, the computing device may receive the compressed delay data and the plurality of pieces of initial focus delay data transmitted by the computer device.

In S1002, the compressed delay data is decompressed to obtain the delay data from the plurality of focuses to the different array elements during the ultrasonic imaging.

In the embodiments of the present disclosure, after decompressing the compressed delay data, the computing device may determine the delay data from the plurality of focuses to the different array elements during the ultrasonic imaging according to focus phase description information and a plurality of pieces of initial focus delay data corresponding to a plurality of array elements obtained by decompression.

In the above data decompression method, a plurality of pieces of initial focus delay data and compressed delay data are received, wherein the compressed delay data is obtained by compressing focus phase description information corresponding to a plurality of array elements, the focus phase description information is determined based on a plurality of pieces of delay data, and the delay data is delay data from a plurality of focuses to different array elements during ultrasonic imaging, and the compressed delay data is decompressed to obtain the delay data from the plurality of focuses to the different array elements during the ultrasonic imaging. According to the method, after the delay data transmitted by the computer device is received, since the compressed delay data is obtained by compressing the focus phase description information corresponding to the plurality of array elements, that is, the phase description information is directly compressed in the compression process, complexity of the calculation process is reduced, thereby reducing complexity of data compression. On the basis of reducing the complexity of data compression, complexity of the data decompression process is also inevitably reduced.

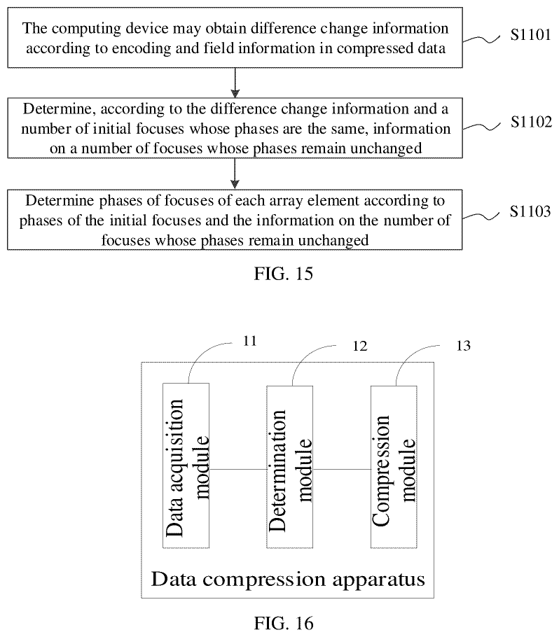

After the computing device receives the data transmitted by the computer device, FIG. 15 is a schematic flowchart of a processing process of a computing device. The processing process may include the following steps. In S1101, the computing device may obtain difference change information according to encoding and field information in compressed data. In S1102, information on a number of focuses whose phases remain unchanged is determined according to the difference change information and a number of initial focuses whose phases are the same. In S1103, phases of the focuses of each array element is determined according to phases of the initial focuses and the information on the number of focuses whose phases remain unchanged. In the manner of compressing the focus phase description information, a data amount during data transmission can be reduced, and a storage space inside the computing device and a data transmission bandwidth can also be saved.

On the basis of the above embodiments, the data decompression method further includes: performing beamforming based on the compressed delay data so as to obtain an ultrasonic image.

Specifically, the computing device firstly performs decompression processing on compressed delay data to obtain delay data from the plurality of focuses to different array elements during ultrasonic imaging, i.e., the decompressed delay data, then performs beamforming on the decompressed delay data to obtain a signal of a target region, and performs signal processing on the signal of the target region obtained after beamforming to obtain an ultrasonic image.

Still further, the data decompression method may further include adjusting at least one of position and parameter of an ultrasound probe based on the obtained ultrasound image, or sending an instruction for adjusting at least one of the position and the parameter of the ultrasound probe to the ultrasound probe.

Specifically, when the ultrasonic image indicates that the focusing of the ultrasonic beam is inaccurate, that is, the result after beamforming indicates that the ultrasonic beam is not focused on an expected target depth or area, the position and/or parameter of the ultrasonic probe may be adjusted, or an instruction for adjusting the position and/or parameter of the ultrasonic probe may be sent to the ultrasonic probe, so as to ensure that the beam can be continuously focused on the target. The parameter of the ultrasonic probe is a parameter applied to the ultrasonic probe when the ultrasonic probe is in operation, for example, including an operating frequency, a transmission power or the like of the ultrasonic probe. Adjusting the position and/or parameters of the ultrasound probe can optimize the effects of beamforming.

The data decompression method according to the present disclosure may be understood as a method for decompressing the compressed delay data obtained according to the data compression method in the foregoing embodiments of the present disclosure.

It is to be noted that when the beamforming algorithm is executed by a computing device such as a field programmable gate array (FPGA), there is only a need to send sampled ultrasonic echo data to the computing unit that executes the beamforming algorithm in sequence according to a sampling clock cycle and determine, from a quantization result, whether the calculation of the current focus still uses the same ultrasonic echo data as the calculation of the previous focus.

It should be understood that, although the steps in the flowcharts as referred to in the embodiments above are shown in sequence as indicated by the arrows, the steps are not necessarily performed in the order indicated by the arrows. Unless otherwise clearly specified herein, the steps are performed without any strict sequence limitation, and may be performed in other orders. In addition, at least some steps in the flowcharts as referred to in the embodiments above may include a plurality of steps or a plurality of stages, and such steps or stages are not necessarily performed at a same moment, and may be performed at different moments. The steps or stages are not necessarily performed in sequence, and the steps or stages and at least some of other steps or steps or stages of other steps may be performed in turn or alternately.

Based on a same inventive concept, embodiments of the present disclosure further provide a data compression apparatus configured to implement the data compression method as referred to above. The implementation solution provided by the apparatus to solve the problem is similar to the implementation solution described in the above method. Therefore, specific limitations in one or more embodiments of the data compression apparatus provided below may be obtained with reference to the limitations on the data compression method above. Details are not described herein.

In an embodiment, as shown in FIG. 16, a data compression apparatus is provided, including: a data acquisition module 11, a determination module 12, and a compression module 13.

The data acquisition module 11 is configured to acquire delay data from a plurality of focuses to different array elements during ultrasonic imaging.

The determination module 12 is configured to determine focus phase description information corresponding to the array elements based on the delay data.

The compression module 13 is configured to compress the focus phase description information to obtain compressed delay data.

In an embodiment, the determination module includes a coefficient acquisition unit and an information determination unit.

The coefficient acquisition unit is configured to acquire, for any one of the array elements, an interpolation coefficient of delay data of all focuses corresponding to the array element.

The information determination unit is configured to determine the focus phase description information corresponding to the array element according to the interpolation coefficient.

In an embodiment, the information determination unit is further configured to quantize the delay data of the focuses according to the interpolation coefficient, and determine a phase of each of the focuses; and determine the phase of each of the focuses to be the focus phase description information of the array element.

In an embodiment, the information determination unit is further configured to acquire delay increment data of adjacent focuses in the array element and acquire phases of the focuses corresponding to the array element; if the delay increment data is within a preset range, determine phase change information of the adjacent focuses according to a mapping relationship between delay increment data and phase change information; and determine the focus phase description information corresponding to the array element based on the phases of the focuses and the phase change information.

In an embodiment, the information determination unit is further configured to acquire, for any one of the array elements, a phase of an initial focus corresponding to the array element; calculate, based on the phase change information and the phases of the focuses, the information on the number of focuses whose phases remain unchanged; and gather the phase of the initial focus and the information on the number of focuses to obtain the focus phase description information of the array element.

In an embodiment, the compression module includes: a sorting unit and a compression unit.

The sorting unit is configured to sort the plurality of pieces of focus number information in each piece of focus phase description information.

The compression unit is configured to compress the sorted focus phase description information to obtain the compressed delay data.

In an embodiment, the data compression apparatus further includes: an information acquisition module, a relationship acquisition module, and a transmission module.

The information acquisition module is configured to acquire initial focus delay data of each of the array elements.

The transmission module is configured to transmit the initial focus delay data and the compressed delay data to a computing device.

In an embodiment, a data decompression apparatus is provided, including:
a receiving module configured to receive a plurality of pieces of initial focus delay data and compressed delay data, wherein the compressed delay data is obtained by compressing focus phase description information corresponding to a plurality of array elements, the focus phase description information is determined based on a plurality of pieces of delay data, and the delay data is delay data from a plurality of focuses to different array elements during ultrasonic imaging; and
a decompression module configured to decompress the compressed delay data to obtain the delay data from the plurality of focuses to the different array elements during the ultrasonic imaging.

The modules in the data compression apparatus and the data decompression module above may be implemented entirely or partially by software, hardware, or a combination thereof. The above modules may be built in or independent of a processor of a computer device in a hardware form, or may be stored in a memory of the computer device in a software form, to facilitate the processor to invoke and perform operations corresponding to the above modules. Exemplarily, the data compression apparatus may be the above computer device, and the data decompression apparatus may be the above computing device.

Specific features of the data compression method and the data decompression method above may be applied to the data compression apparatus and the data decompression module respectively. Details are not described herein again.

In an embodiment, a computer device is provided, including a memory and a processor. The memory stores a computer program. The processor, when executing the computer program, implements content in any one of the embodiments in the data compression method.

In an embodiment, a computer-readable storage medium is provided, having a computer program stored therein. Content in any one of the embodiments of the data compression method above is implemented when the computer program is executed by a processor.

In an embodiment, a computer-readable storage medium is provided, having a computer program stored therein. Content in any one of the embodiments of the data decompression method above is implemented when the computer program is executed by a processor.

In an embodiment, a computer program product is provided, including a computer program. Content in any one of the embodiments of the data compression method above is implemented when the computer program is executed by a processor.

It is to be noted that user information (including, but not limited to, user equipment information, user personal information, and the like) and data (including, but not limited to, data for analysis, stored data, displayed data, and the like) as referred to in the present disclosure are information and data authorized by the user or fully authorized by all parties.

Those of ordinary skill in the art may understand that some or all procedures in the methods in the foregoing embodiments may be implemented by a computer program instructing related hardware, the computer program may be stored in a non-transitory computer-readable storage medium, and when the computer program is executed, the procedures in the foregoing method embodiments may be implemented. Any reference to the memory, database, or other media used in the embodiments provided in the present disclosure may include at least one of a non-transitory memory and a transitory memory. The non-transitory memory may include a read-only memory (ROM), a magnetic tape, a floppy disk, a flash memory, an optical memory, a high-density embedded non-transitory memory, a resistive random access memory (ReRAM), a magnetoresistive random access memory (MRAM), a ferroelectric random access memory (FRAM), a phase change memory (PCM), a graphene memory, and the like. The transitory memory may include a random access memory (RAM) or an external cache memory. By way of illustration instead of limitation, the RAM is available in a variety of forms, such as a static random access memory (SRAM) or a dynamic random access memory (DRAM). The database as referred to in the embodiments provided in the present disclosure may include at least one of a relational database and a non-relational database. The non-relational database may include a blockchain-based distributed database, and the like, but is not limited thereto. The processor as referred to in the embodiments provided in the present disclosure may be a general-purpose processor, a central processing unit, a graphics processor, a digital signal processor, a programmable logic device, a data processing logic device based on quantum computing, and the like, but is not limited thereto.

The technical features in the above embodiments may be randomly combined. For concise description, not all possible combinations of the technical features in the above embodiments are described. However, all the combinations of the technical features are to be considered as falling within the scope described in this specification provided that they do not conflict with each other.

The above embodiments only describe several implementations of the present disclosure, and their description is specific and detailed, but cannot therefore be understood as a limitation on the patent scope of the present disclosure. It should be noted that those of ordinary skill in the art may further make variations and improvements without departing from the conception of the present disclosure, and these all fall within the protection scope of the present disclosure. Therefore, the protection scope of the present disclosure should be subject to the appended claims.

What is claimed is:

1. A data compression method, comprising:
acquiring delay data from a plurality of focuses to different array elements during ultrasonic imaging;
determining focus phase description information corresponding to the array elements based on the delay data; and
compressing the focus phase description information corresponding to the array elements to obtain compressed delay data.

2. The method according to claim 1, wherein determining the focus phase description information corresponding to the array elements based on the delay data comprises:
acquiring, for any one of the array elements, an interpolation coefficient of delay data of all focuses corresponding to the array element; and
determining the focus phase description information corresponding to the array element according to the interpolation coefficient.

3. The method according to claim 2, wherein acquiring, for any one of the array elements, the interpolation coefficient of the delay data of all the focuses corresponding to the array element comprises:
pre-determining, according to required image resolution and an error size, the interpolation coefficient of the delay data of all the focuses corresponding to the array element.

4. The method according to claim 2, wherein acquiring, for any one of the array elements, the interpolation coefficient of the delay data of all the focuses corresponding to the array element comprises:
acquiring historical delay data most similar to the delay data of all the focuses corresponding to the array element, and determining an interpolation coefficient corresponding to the historical delay data to be the interpolation coefficient of the delay data of all the focuses corresponding to the array element.

5. The method according to claim 2, wherein the focus phase description information comprises phases of the focuses; and
determining the focus phase description information corresponding to the array element according to the interpolation coefficient comprises:
quantizing the delay data of the focuses according to the interpolation coefficient, and determining a phase of each of the focuses; and
gathering the phase of each of the focuses to obtain the focus phase description information of the array element.

6. The method according to claim 5, wherein gathering the phase of each of the focuses to obtain the focus phase description information of the array element comprises:
taking a phase of a starting focus of the array element as an initial phase, and acquiring phases of other focuses after quantization;
generating a table indicating the phases of the plurality of focuses in order of the focuses; and
taking content of the table as the focus phase description information of the array element.

7. The method according to claim 2, wherein the focus phase description information comprises information on a number of focuses whose phases remain unchanged; and
determining the focus phase description information corresponding to the array element according to the interpolation coefficient comprises:
acquiring delay increment data of adjacent focuses in the array element, and acquiring phases of the focuses corresponding to the array element;
if the delay increment data is within a preset range, determining phase change information of the adjacent focuses according to a mapping relationship between delay increment data and phase change information; and
determining the focus phase description information corresponding to the array element based on the phases of the focus and the phase change information.

8. The method according to claim 7, wherein acquiring the delay increment data of the adjacent focuses in the array element comprises:
calculating a difference between the delay data of two adjacent focuses in the array element, and taking the difference as the delay increment data of the adjacent focuses in the array element.

9. The method according to claim 7, wherein the preset range is greater than (N−1)/N*T and less than T, where T denotes an original sampling cycle of the delay data, and N denotes the interpolation coefficient.

10. The method according to claim 7, wherein determining the focus phase description information corresponding to the array element based on the phases of the focuses and the phase change information comprises:
sequentially determining, according to a focus sampling order, a number of focuses whose phases do not change, and gathering, according to the focus sampling order, the number of focuses whose phases do not change, to obtain the focus phase description information corresponding to the array element.

11. The method according to claim 7, wherein determining the focus phase description information corresponding to the array element based on the phases of the focuses and the phase change information comprises:
acquiring, for any one of the array elements, a phase of an initial focus corresponding to the array element;
calculating, based on the phase change information and the phases of the focus, the information on the number of focuses whose phases remain unchanged; and
gathering the phase of the initial focus and the information on the number of focuses to obtain the focus phase description information corresponding to the array element.

12. The method according to claim 11, wherein gathering the phase of the initial focus and the information on the number of focuses to obtain the focus phase description information corresponding to the array element comprises:
putting the phase of the initial focus first in the focus phase description information; and
arranging, after the phase of the initial focus, the information on the number of focuses in order of sampling time, and gathering the arranged information, to obtain the focus phase description information of the array element.

13. The method according to claim 11, wherein the focus phase description information comprises a plurality of pieces of focus number information; and
compressing the focus phase description information to obtain the compressed delay data comprises:
sorting the plurality of pieces of focus number information in each piece of focus phase description information; and
compressing the sorted focus phase description information to obtain the compressed delay data.

14. The method according to claim 1, further comprising:
acquiring initial focus delay data of each of the array elements; and
transmitting the initial focus delay data and the compressed delay data to a computing device.

15. A data decompression method, comprising:
receiving a plurality of pieces of initial focus delay data and compressed delay data; the compressed delay data being obtained by compressing focus phase description information corresponding to a plurality of array elements; the focus phase description information being determined based on a plurality of pieces of delay data; and the delay data being delay data from a plurality of focuses to different array elements during ultrasonic imaging; and
decompressing the compressed delay data to obtain the delay data from the plurality of focuses to the different array elements during the ultrasonic imaging.

16. The method according to claim 15, further comprising:
performing beamforming based on the obtained delay data from the plurality of focuses to the different array elements during the ultrasonic imaging to obtain an ultrasonic image.

17. A data compression apparatus, the apparatus comprising a memory and a processor, the memory storing a computer program, and the processor, when executing the computer program, implementing steps of the data compression method according to claim 1.

18. A data decompression apparatus, the apparatus comprising a memory and a processor, the memory storing a computer program, and the processor, when executing the computer program, implementing steps of the data decompression method according to claim 15.

19. A non-transitory computer-readable storage medium, having a computer program stored therein, steps of the data compression method according to claim 1 being implemented when the computer program is executed by a processor.

20. A non-transitory computer-readable storage medium, having a computer program stored therein, steps of the data decompression method according to claim 15 being implemented when the computer program is executed by a processor.
